(19) United States
(12) Patent Application Publication
(10) Pub. No.: US 2004/0073180 A1
(43) Pub. Date: Apr. 15, 2004

(54) Absorbent article for men

(76) Inventor: Kenneth Strannemalm, Floda (SE)

Correspondence Address: BURNS, DOANE, SWECKER & MATHIS, L.L.P., P.O. Box 1404, Alexandria, VA, 22313-1404, US

(21) Appl. No.: 10/612,370

(22) Filed: Jul. 3, 2003

Related U.S. Application Data
(60) Provisional application No. 60/393,477, filed on Jul. 5, 2002.

(51) Int. Cl. A61F 5/44; A61F 13/15; A61F 13/20

(52) U.S. Cl. 604/349; 604/385.27; 604/385.14; 604/395; 604/398

(57) ABSTRACT

A pants-shaped absorbent product, such as incontinence pants or pull-ups, intended for males, has a front section, a rear section, a crotch section, an elastic waist region, an absorbent element intended to cover the penis of the user, and a liquid-tight outer layer. The front section has an elastic member, which enables the front section of the product to be pulled down during use, counter to the action of the elastic member, to a position in which the upper limit edge of the front section is situated below the penis of the user. The absorbent element is configured with one or more deformation zones, which enable parts of the absorbent element to be drawn along when the front section of the product is pulled down. The front section of the product and the absorbent element are arranged to be returned by the elastic member to their original usage position.

### CROSS REFERENCE TO RELATED APPLICATION

This application claims priority under 35 U.S.C. § 119(e) to U.S. Provisional Application No. 60/393,477 entitled ABSORBENT ARTICLE FOR MEN and filed on Jul. 5, 2002, the entire content of which is hereby incorporated by reference.

### BACKGROUND

1. Field of the Invention

The present invention relates to a pants-shaped absorbent product, such as incontinence pants or pull-ups, intended for males. The absorbent product comprises a front section, a rear section, a crotch section, side sections, which are intended to connect the front section and the rear section in the lateral direction and which comprise hip sections, an elastic waist region, and an absorbent element which, when the article is in use, is intended to at least cover the penis of the user. The absorbent article further comprises a liquid-tight outer layer, which is intended to enclose the absorbent element at least on the side thereof facing away from the user when the product is worn.

2. Background of the Invention

Absorbent disposable products for absorbing urine have developed a lot since entering into more general use during the 1960's and 1970's. Because they are disposable products, it is necessary for them to be produced and sold at a very low price. At the same time, it is important that the functioning of the products is good and reliable. Good fit and comfort are also important characteristics. The first disposable diapers were constituted by two-piece products, outer pants made of plastic, which were intended for reuse, and a rectangular absorbent insert article for single usage. The absorbent material in these insert articles was initially constituted by cellulose. Later, better absorption material, consisting of so-called cellulose fluff, was developed. The fit and comfort of these early diapers was poor. The products were cumbersome and uncomfortable for the user. In the late 1970's, the first so-called complete disposable diapers were born, i.e., diapers in which the absorption bodies were integrated with a liquid-tight outer layer. The absorption materials were developed and became better, which meant that the absorption bodies could be better adapted to the body shape of the user. Hourglass-shaped absorption cores having a narrower crotch section between the two end sections are now dominant. The trend has also been towards thinner products, which have been made possible by the mixing of so-called superabsorbent material into the absorption body. There are numerous reasons why thinner and also smaller absorption bodies are sought. A thinner and smaller absorption body is more comfortable and more discrete, which is important for adult incontinent users. A reduction in volume is also economically very important, since less storage space is needed, transportation is made easier and less shelving is needed in the shops. The last-named is important for shop economics. If products can be developed which require less shop space than competitors' products, a considerable competitive advantage is gained. Moreover, there is increased pressure from the authorities to use as little material as possible, especially in disposable articles, to lessen environmental pollution.

The smaller the absorption bodies, the more important it is that the absorption bodies should land in the right place directly in front of the genitals of the user and should remain in place during use, even when the user is very active and moving a lot. Consumers are also becoming increasingly demanding about discretion, comfort, and reliability. The requirements that the absorption body will end up correctly positioned when it is donned and will thereafter be held in the right place have therefore increased the need for the product to be securely fixed on the body and for it to respond closely to the body when the user moves, at the same time as requirements have increased for the product always to land in the right place when the article is applied to the user. This has led to the development of so-called pull-ups, which have elastic sections for enhanced fit and comfort and enhanced responsiveness to the movements of the user compared with conventional absorbent products.

An early patent publication relating to disposable-type pull-ups is GB 2 112 267-A. This 1983 publication shows primitive pull-ups which never became a commercial product. Not until the 1990's did absorbent products having a pants-like shape and design become a commercial success. Pants-like products now exist in the form of children's diapers and adults' diapers.

Pull-ups which have hitherto been commercially available have been configured, in principle, like conventional diapers, having a front section and a rear section and an intermediate crotch section, the front and rear section being mutually joined.

Known pull-ups which are available on the market work satisfactorily for women. As far as male incontinence is concerned, there are so far no pull-ups which are satisfactory in every respect.

A major problem for male incontinence sufferers who do not have any movement-related problems is that public toilets for men have urinals, the use of which requires the user to have pants with a fly. It is naturally inconceivable for a man, in connection with a toilet visit at a urinal, to pull down the whole of the pull-ups and thereby reveal private and embarrassing problems to those around him. The consequence of this is that even relatively minor incontinence problems become very embarrassing for men in everyday life. Visits to sports arenas and other major events are, in practice, almost impossible for men with incontinence problems. Correspondingly, so-called “training pants”, which are used when children have to be weaned off diapers, also to some extent represent a problem for small boys. Over the years, there have been a host of solutions proposed to the above-mentioned problems. Common to all these proposed solutions is that the diaper or pull-up has been provided with some form of opening, which is intended to serve as a fly opening.

In U.S. Pat. No. 4,326,302, a two-piece product is described, having pants provided with a pocket for receiving an absorption element, which, when the pants are used, is situated over the genital region of the user. The pants are provided with an opening next to the pocket, which opening is intended to serve as a fly opening. This solution does not work satisfactorily, since the absorption element has to be firmly displaced in the lateral direction in order for the user to get the opportunity to fulfil his needs via the opening. In addition, it is awkward and time-consuming to bring the absorption element into the correct position following use of the opening.

In U.S. Pat. No. 4,589,877, an insert is described in which the absorption element is divided and in which the parts are fitted in an overlapping arrangement with a fly opening between the parts. A problem with this design is that the insert in the region of the overlap is bulky and cumbersome. Moreover, a product according to this publication is difficult to produce. There is also a risk of leakage through the fly opening.

In U.S. Pat. No. 4,944,733, a diaper is described which has been provided with an opening extending from the waist region to the crotch region of the diaper. The opening is constituted by an elongated slot and this opening is closed by sections on both sides of the opening being fitted in overlapping arrangement. The diaper is provided with adhesive fasteners which hold the opening in closed position. A fundamental drawback with this diaper design is the said adhesive fasteners, which are subjected to high stresses when the diaper is worn and when the fasteners have to be undone to free the opening. Production of such products is troublesome and costly.

In U.S. Pat. No. 5,843,065, an incontinence diaper for men is described with an opening situated directly in front of the penis of the user and through which an absorption pad situated inside the opening can be folded out for access to the penis inside the slot. In practice, this design is impractical especially when the absorption pad has to be returned to its intended place inside the opening following a toilet visit. An incontinence diaper according to U.S. Pat. No. 5,843,065 is, moreover, both awkward and expensive to produce.

No satisfactory solution to the problem has hitherto been presented and there are presently no products whatsoever on the market despite the fact that the problem is old and well known.

### OBJECTS AND SUMMARY

As a result of the present invention, an improved pants-shaped absorbent product has been produced, which product wholly eliminates or reduces the abovementioned problems.

The present invention provides for a product wherein the front section has at least one elastic member, which, during product usage, enables the front section of the product to be pulled down, counter to the action of the said elastic member, to a position in which the upper limit edge of the front section in the middle region of the front section is situated below the penis of the user, at the same time as at least the hip sections of the product are arranged to be held in place around the waist of the user, wherein the absorbent element is configured with one or more deformation zones, which enable those parts of the absorbent element which are situated above and over the penis of the user during product usage to be drawn down together with the rest of the front section when the front section of the product is pulled down, and wherein the front section of the product, together with the absorbent element, are arranged to be returned by the elastic member to their original usage position.

In the product according to an embodiment of the present invention, no special fly opening is needed on the absorbent product. Rather, a user introduces his hand through the fly opening in the outer pants and, by a simple maneuver, guides down the front section of the absorbent product in order to free the penis.

According to one embodiment, the elastic member, over the whole of its working range, has an essentially constant tensile stress when stretched, which tensile stress is in the same order of magnitude as the tensile stress in the elastic waist region.

According to one embodiment, the elastic member is constituted by a section of the elastic waist region. According to one embodiment, the section extends over the front section between the hip sections.

According to one embodiment, the elastic waist region extends over the whole of the front section, rear section and side sections of the product and in that the said section of the elastic waist region which serves as the elastic member has higher elastic stretchability than the rest of the elastic waist region.

According to a further embodiment, the absorbent element is of thinner and more flexible configuration in the deformable zone compared with the rest of the absorbent element.

According to another embodiment, the absorbent element incorporates deformable zones in the form of fold notches for the absorbent element, along which fold notches the product is bent when the front section of the product is pulled down.

According to a further embodiment, the fold notches are constituted by recesses, through-slots or compression lines in the absorbent element.

According to another embodiment, the absorbent element is constituted by at least two absorption bodies of different extent, in that only one of these extends into the deformation zone and in that the absorption body extending into the deformation zone has little or no inherent stiffness.

According to another embodiment, the front section of the product, at the waist region, has, when viewed from the crotch region, a concave recess between the said hip sections, whereby the front section of the product, during usage, acquires a low pants cut, which, compared with a straight cut, reduces the amount by which the upper limit edge of the product in the middle region of the front section needs to be pulled down in order to bring the said limit edge into position below the penis of the user.

According to one embodiment, the hip sections of the product are provided with friction members, which are arranged to help the product as a whole to be held in place on the user as the front section of the product is pulled down.

According to a further embodiment, the elastic waist section is formed from an elastic first piece which, in the extended state, is essentially rectangular and which is intended to partially surround the trunk of the user and form the rear section and side sections of the pants-like product. A second piece, incorporated in the product, is configured to form the front section and crotch section of the pants-like product, and is elongated with two opposing end edges and two opposing longitudinal edges. The width of the second piece, at least at the crotch section, is less than the length of the first piece. The second piece is arranged with its longitudinal direction perpendicular to the longitudinal direction of the first piece and is connected by a first end section to the one longitudinal edge section of the first piece and centrally on this, wherein the one end section of the first piece is connected to a first side edge section of the second piece, and wherein the second end section of the first piece is correspondingly connected to a second side edge section of the second piece, and the absorbent element is fitted, in its entirety, on the second piece.

By virtue of the fact that the whole of the rear section is also elastic and, together with the elastic side sections, forms a single elastic continuous first piece, the pants as a whole are very responsive to body movements. Local disturbances are absorbed and smoothed out by the continuous elastic piece and are not transmitted to the less resilient parts of the front section and crotch section situated directly in front of the genitals of the user. Compared with conventional diapers and previously known pants-shaped absorbent products, superior fit and comfort are obtained. The greater part of the pants-like garment remains totally plain. The seams needed are fitted at the transition between the front section and the first piece and at the transition between the first and second piece at the crotch section. These seams end up in places which are not subjected to any great pressure during product usage and there is less risk of chafing and pressure sores caused by seams on the pants-like product compared with known pull-ups currently available.

The configuration of the pants sheath from only two part-pieces and in which the absorbent element is situated entirely on the second part-piece offers increased freedom in the choice of production method compared with previously known pants-shaped products.

According to one embodiment, the connected side edge sections and end sections of the said first and second pieces are fitted, prior to connection, in overlapping arrangement with the inner side of an overlapping section placed against the outer side of an overlapped section.

According to another embodiment, the absorbent element extends in its longitudinal direction over the whole of the crotch section and slightly up over the front section in the direction of the upper side edge of the second piece, during product usage.

According to one embodiment, the said elastic member is constituted by an elastic non-woven or an elastic film.

According to another embodiment, the said elastic member is constituted by elastic tape or threads.

According to another embodiment, the absorbent product is constituted by a two-piece product having an absorbent element in the form of a detachable insert intended to be fitted inside pants incorporated in the product. The insert has a rear liquid-tight layer provided with fastening members by means of which the insert is detachably anchored in the pants. The insert, when the front section of the product is pulled down, is arranged to be held in place relative to the pants by means of the said fastening members.

According to a further embodiment, a number of separate absorption bodies are fitted adjacent to one another in the lateral direction, extend in the longitudinal direction of the product in the direction from the front section to the rear section, and are separable in the lateral direction and longitudinal direction counter to the action of transverse elastic, whereby the separate absorption bodies can be mutually displaced when the front section of the product is pulled down.

### BRIEF DESCRIPTION OF THE FIGURES

The invention will be described in greater detail below with reference to illustrative embodiments shown in the appended drawings, in which:

FIG. 1 shows diagrammatically a phase in the development of an absorbent product according to a first embodiment.

FIG. 2 shows diagrammatically in flat form an assembled product according to the first embodiment.

FIG. 3 shows in perspective the product according to FIG. 2.

FIG. 4 shows a section along the line IV-IV in FIG. 1.

FIG. 5 shows diagrammatically in flat form an example of an absorbent element incorporated in an absorbent product according to the invention.

FIG. 6 shows diagrammatically in flat form another example of an absorbent element incorporated in an absorbent product according to the invention.

FIG. 7 shows diagrammatically a phase in the development of an absorbent product according to a second embodiment.

FIG. 8 shows in flat form an assembled product according to the second embodiment.

FIG. 9 shows a longitudinal section through a further example of an absorbent element for use in an absorbent product according to the invention.

FIG. 10 shows in plan view another example of an absorbent element for use in an absorbent product according to the invention.

FIG. 11 shows a broken section through an example of a deformation zone in an absorbent element in an absorbent product according to the invention.

FIG. 12 shows in plan view an insert incorporated in a two-piece product according to the invention.

FIG. 13 shows a further embodiment of an absorbent product according to the invention.

FIG. 14 shows diagrammatically another illustrative embodiment of an absorbent product according to the invention.

FIG. 15 shows diagrammatically a somewhat modified embodiment in relation to the embodiment according to FIG. 1.

### DETAILED DESCRIPTION OF THE PREFERRED EMBODIMENTS

As can be seen from FIG. 1, the product in the embodiment according to FIG. 1 comprises a first piece 1. This is elastically stretchable and is shown in FIG. 1 in flat and evenly extended state, in which the elastic piece 1 is essentially rectangular. The elastic first piece 1 can be formed from conventional materials familiar to the person skilled in the art, such as woven elastic materials, elastic non-wovens, or elastic films. The elastic piece is stretchable for a user when putting on the product and it fits tight with suitable tightening around the user when the article is worn. The tightening is adjusted, of course, by means of size and elastic stretchability.

The product incorporates a second piece 2, which, as can be seen from FIGS. 1 and 4, comprises an outer layer 3, an inner layer 4 and an absorbent element 5 fitted between these. In the embodiment shown in FIG. 1, this is in flat form essentially triangular. The choice of material in the absorption element is not critical but can be chosen from materials or material combinations familiar to the person skilled in the art. For example, the absorbent element can be made up of cellulose fluff with superabsorbent material in powder or fiber form mixed in with this. The outer layer 3 can consist of, for example, a polyethylene film of a type conventionally used for absorbent products. A liquid-tight film in combination with an outer fiber layer is suitable if an absorbent product having a more textile-like appearance is sought. The inner layer 4 can be formed from a liquid-permeable non-woven material. The second piece 2 is elongated and is fitted with its longitudinal direction perpendicular to the longitudinal direction of the first piece 1. The second piece 2 is connected by an end section 6 to the one longitudinal edge section 7 of the first piece 1 and centrally on this. The connection can be realized, for example, by the use of bonding agent, heat bonding or ultrasonic bonding.

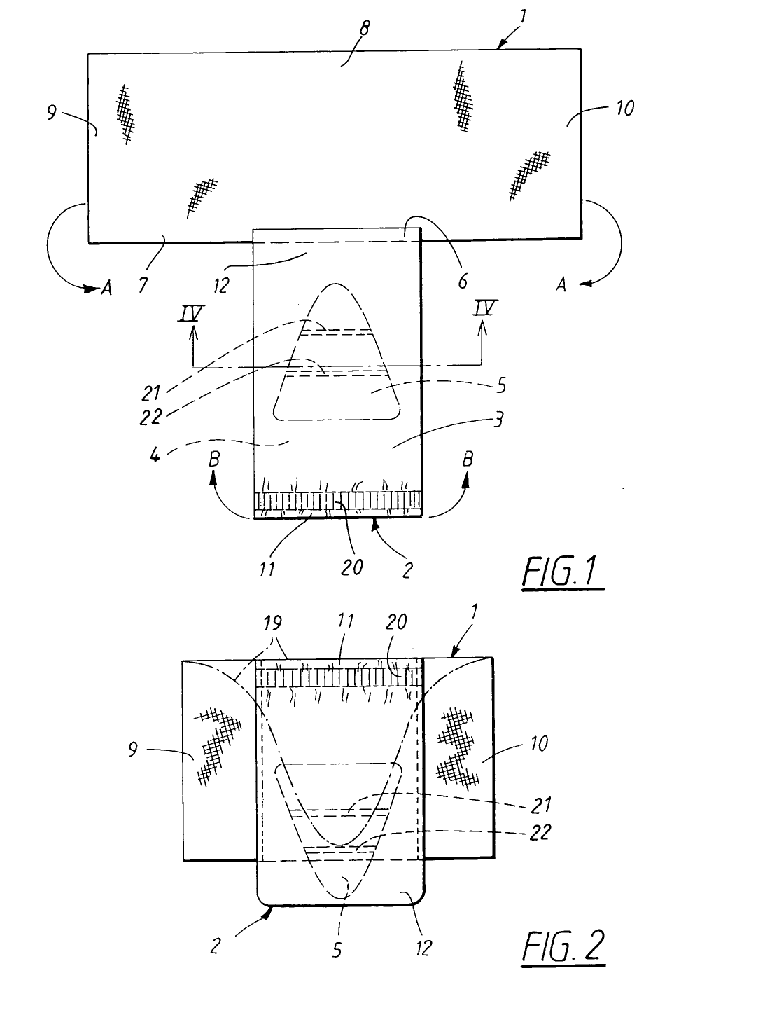

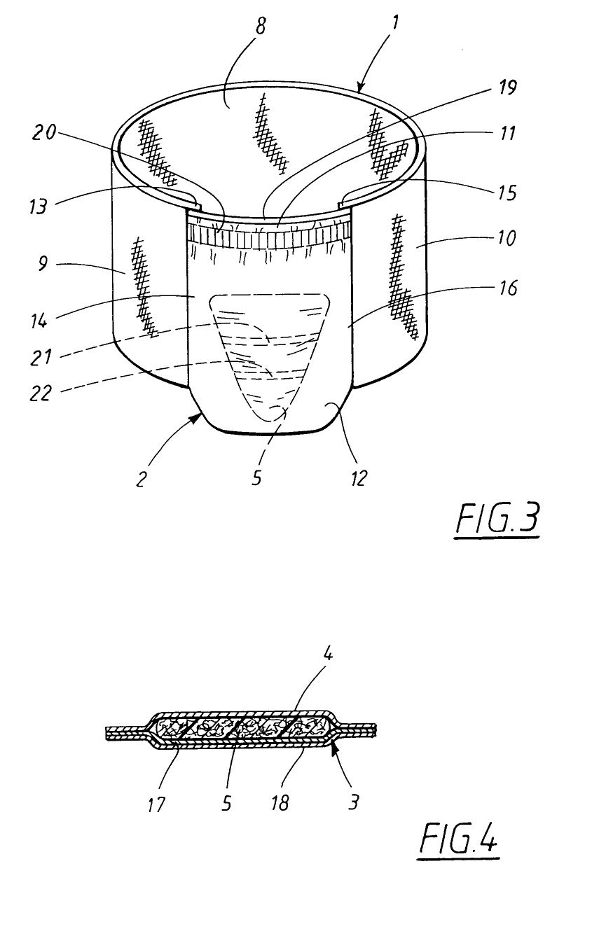

In FIG. 1, the arrows A and B are used to illustrate how the first and second pieces are folded for the formation of the pants-shaped absorbent product shown in FIGS. 2 and 3. The elastic piece 1 is folded in according to the shown arrows A to form the rear section 8 and side sections 9, 10 of the products, whereas the second piece is folded upwards according to the arrows B to form the front section 11 and crotch section 12 of the product.

As can clearly be seen from FIG. 3, the one end section 13 of the first piece is fitted in slightly overlapping arrangement with a first side edge section 14 of the second piece and the second end section 15 of the first piece is correspondingly fitted in slightly overlapping arrangement with a second side edge section 16 of the second piece. These overlapping parts are mutually connected, for example, by means of bonding agent, heat bonding or ultrasonic bonding. Alternatively, the front section and the crotch section can be detachably fitted along the said overlapping sections 13, 14 and 15, 16 respectively. A detachable connection of this kind can be constituted, for example, by a hook member (not shown), whereby the pants-like product can be opened and subsequently closed up again with the same fit and tightening.

In FIG. 4, the thickness of the second piece has been exaggerated for the sake of clarity. In the figure, an outer layer 3 consisting of a laminate of a plastics film 17 and an outermost non-woven layer 18 is shown. The inner layer 4 can be constituted by a liquid-permeable non-woven of a type familiar to the person skilled in the art.

The front section 11 is provided, up to close to its upper limit edge 19, with an elastic member 20 in the form of an elastic tape. This is fitted pretensioned on any of the layers 3, 4 and 5 incorporated in the front section. The elastic member, over the whole of its working range, expediently has an essentially constant tensile stress when stretched, which tensile stress is expediently in the same order of magnitude as the tensile stress in the elastic first piece 1. An upper section of the elastic first piece 1 forms, together with the elastic member 20, a circumferential elastic waist region.

The elastic member 20 expediently has higher elastic stretchability than the elastic first piece 1, which means that the elastic member has greater elastic travel than the elastic first piece upon the expansion of the elastic waist region. The aim of this difference in elastic travel when subjected to the same tensile force is that the front section of the pants-like product will be able to be pulled down without risk of the product being pulled down, in its entirety, from the waist region of the user. During use, the front section of the pants will be able to be pulled down in front counter to the action of the said elastic member 20 to a position in which the upper limit edge 19 of the front section in the middle region of the front section is situated below the penis of the user. The aim is that the user, when visiting a urinal, without any problem whatsoever and with one hand, will be able to pull down just the front section to the position, substantially corresponding to that indicated by dashed lines in FIG. 2, in which the dashed lines 19 are the upper limit edge of the front section after this has been pulled down. This will be able to be effected without the pants being pulled down in their entirety. In the embodiment shown in FIGS. 1-4, the hip sections of the product on the side pieces 9 and 10, as can be seen from FIG. 2, when the front section of the product is pulled down, will essentially be unaffected and will remain in fitted position on the hip bone of the user.

In order that the pants-like product can be pulled down at the front, as intended, in order to bare the penis of the user, it is also required that the absorbent element 5 is configured such that it can be displaced or deformed in a controlled manner without becoming damaged.

The absorbent element 5, so as not to give rise to irritating chafing in the crotch of the user, has been configured such that it tapers in the direction pointing rearwards during product usage. A complication with this tapered configuration is that the absorbent element is at its narrowest in the section which, during product usage, is situated substantially directly in front of the genitals of the user, in which section the absorbency requirement is greatest. In order, as far as practicable, to avoid the risk of leakage in the narrower section of the absorbent element, according to one embodiment in which superabsorbent material in particle or fiber form is incorporated in the absorbent element, it can be expedient for the superabsorbent material to be applied in increasing concentration in the said direction of taper. A shape of the absorbent element 5 which tapers in the direction of the crotch section is also more discrete than, for example, a rectangular absorption body which is folded in an uncontrolled manner in the crotch and is more bulky, whilst, at the same time, formed folds can conduct received urine in the lateral direction out of the absorbent product and cause urine leakage.

In the illustrative embodiment shown in FIGS. 1-4, the absorbent element is provided with deformable zones in the form of fold notches 21, 22, along which the absorbent element is bent when the front section of the product is pulled down. The absorbent element 5 is firstly bent at the fold notch 21 and thereafter, as the product continues to be pulled down, at the fold notch 22. At least the upper fold notch 21 is configured such that the absorbent element here bends out from the user. Once the front section of the product has been fully pulled down, the absorbent element assumes a temporary Z-like shape in longitudinal section. When the user has fulfilled his needs and lets go of the pants and the elastic member 20, the front section of the pants and the absorbent element 5 return to their original state of use through the action of the elastic in the elastic member 20.

In the embodiment shown in FIGS. 1-4, the deformation zones have the form of transverse fold notches. This allows the product to be successfully pulled down and returned to its original position without residual deformation. If a different down-fold is desired, during which the absorbent element, in the course of being folded down, requires less space out from the user, the fold notches can be arranged with different extent, for example such that the fold notches acquire a U-like or V-like shape with the arms of the said U or V-shape essentially parallel with the side edges of the downward-tapered absorbent element.

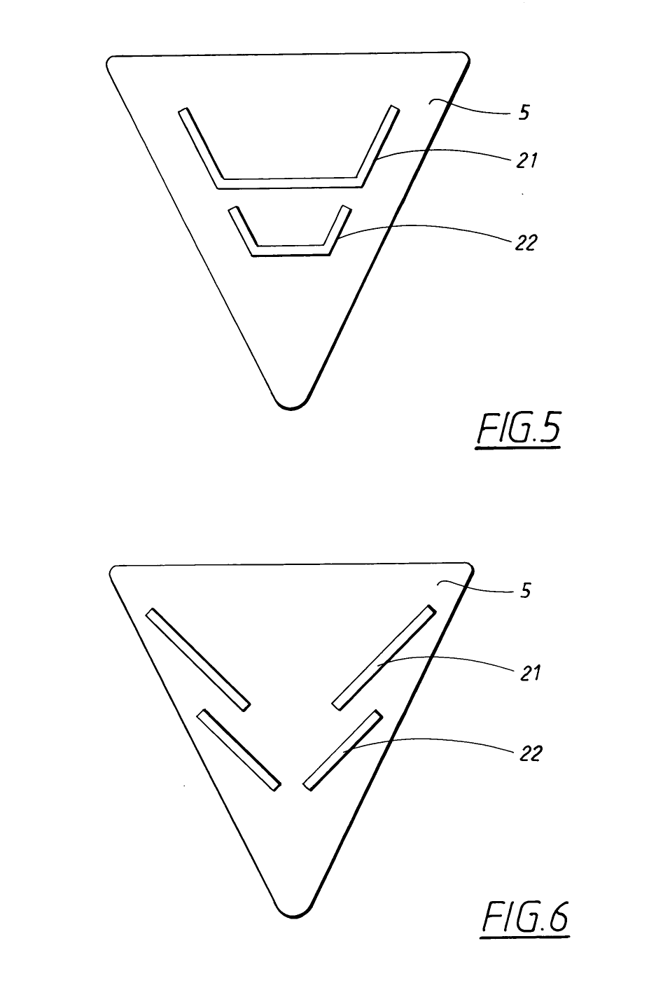

In FIGS. 5 and 6, two examples are shown of U-shaped or V-shaped fold notches 21, 22 arranged on an absorbent element 5. The number of fold notches can, of course, be more than two in the vertical direction, depending on how the absorption body is wished to be deformed when the front section is pulled down.

The fold notches can also be in the form of relatively wide recesses, arranged, otherwise, with the same extent as shown in FIGS. 1-6.

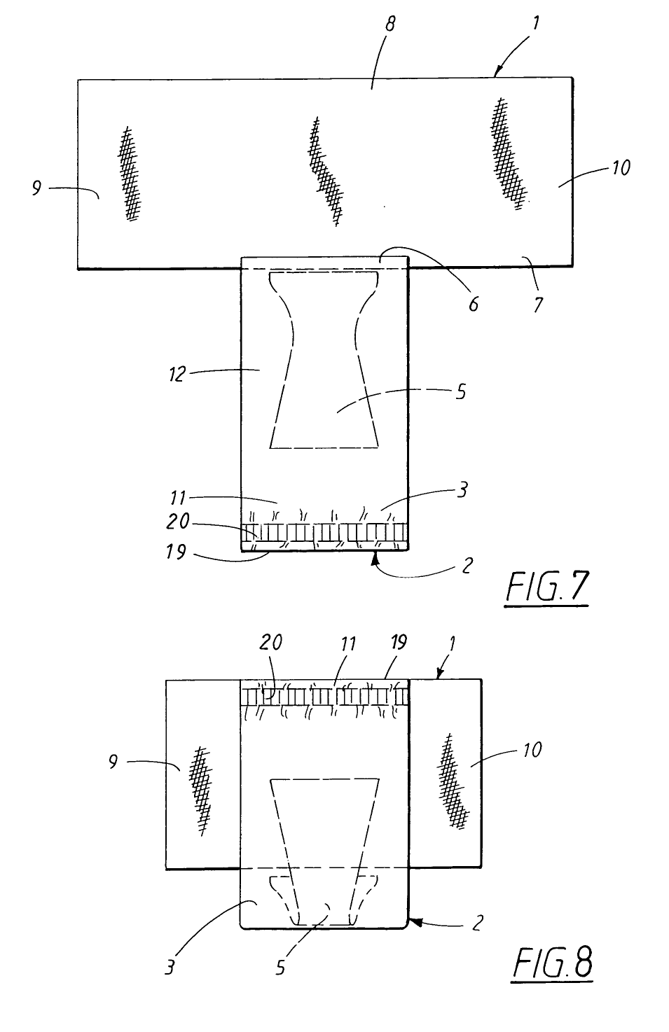

In the embodiments according to FIGS. 1-6, the absorbent element 5 is essentially triangular in shape and is situated wholly on the user in front, during product usage, and does not cover the whole of the crotch section of the user. The absorbent element can, of course, have a different shape, for example, essentially hourglass-shaped as shown in FIGS. 7 and 8. In FIGS. 7 and 8, the same reference notations have been used compared to similar components in the embodiments according to FIGS. 1-4.

In FIG. 9, an alternative realization of an absorbent element 5 is shown, having a deformable zone 30. The absorbent element is shown diagrammatically in cross section in an imaginary usage position. Other details in the absorbent product have not been included, for the sake of simplicity. The deformable zone has here been produced by virtue of it being substantially thinner and having lower bending resistance than the rest of the absorbent element.

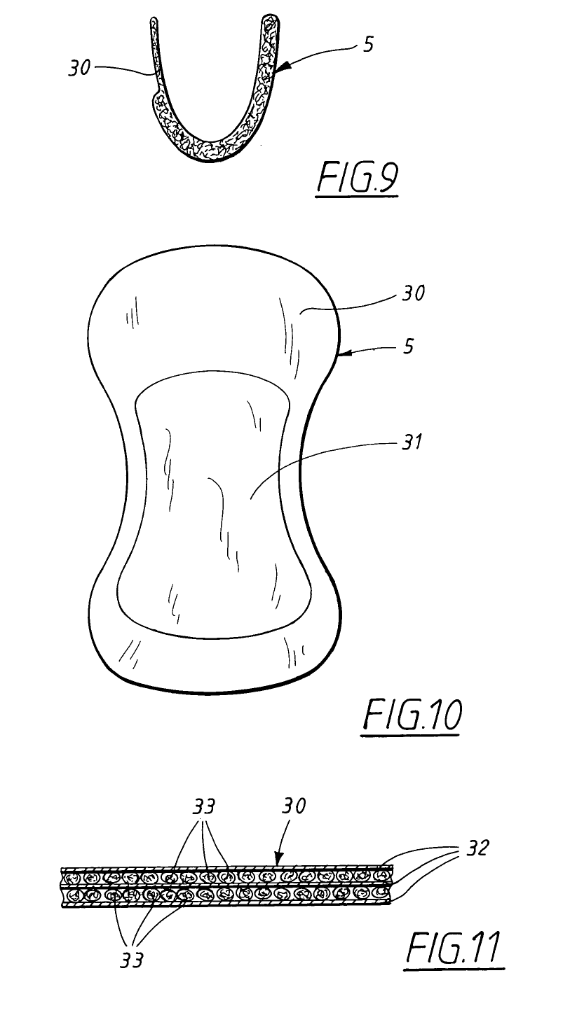

In FIG. 10 a similar illustrative embodiment of an absorbent element 5 having a deformable zone 30 is shown in flat form. The absorbent element here comprises two separate absorption bodies fitted one on top of the other, the one with greater extent being thinner and having substantially lower bending resistance than the two absorption bodies together.

An illustrative embodiment of an absorption body serving as a deformation zone is shown in FIG. 11 in diagrammatic representation and on enlarged scale in the form of a part-section through the absorption body. The absorption body, which has here been denoted by 30, comprises a number of material layers 32 of non-woven material or tissue with a bed of highly absorbent particles 33 between neighbouring material layers. An absorption body of this type, compared with other conventional absorption bodies, for example made of compressed fluff substance, has little or no inherent stiffness and can therefore be easily deformed in connection with a front section of an absorbent product according to the invention being pulled down. It is expedient for the highly absorbent particles to be bound to the material layers to prevent the absorption body from being permanently deformed as a result of displacement and agglomeration of the particles when the front section of the absorbent product is pulled down by the user. This bonding will, of course, not be such that the absorption body is substantially stiffened compared with when the particles are totally unbound.

The absorbent product according to the invention can be constituted by a two-piece product having an absorbent element in the form of a detachable insert, which is intended to be fitted inside pants incorporated in the product. An illustrative embodiment of an insert is shown in FIG. 12. The insert incorporates an absorbent element 5, which is provided with essentially V-shaped deformation zones 21, 22. The absorbent element is enclosed in a sheath comprising a liquid-tight layer 35 on that side which faces away from the user during use and a liquid-permeable layer 36 on the opposite side. The two layers extend in the lateral direction beyond the absorbent element and are there mutually joined. In the shown illustrative embodiment, the insert is provided with longitudinal edge elastic 37 for the formation of leakage protection in the lateral direction of the product. For the marking of the end which is to be fitted facing the front, the shown insert containing the layers has a recess 38 in this end. The insert is intended to be fitted in outer pants (not shown), provided with an elastic member of the type described in connection with the embodiments according to FIGS. 1-4. The liquid-tight layer is expediently provided on its outer side with one or more fastening member(s) (not shown) for detachable anchorage to the outer pants, so that the insert sits fixedly at the intended place inside the pants during product usage and when the pants and the deformable section of the product are pulled down by the user during a toilet visit.

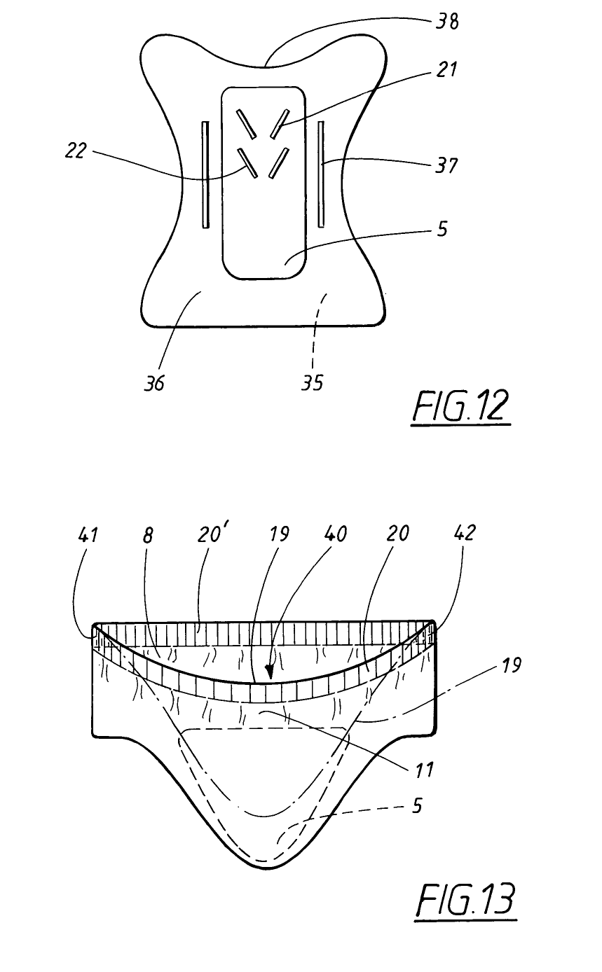

In the illustrative embodiment of an absorbent product according to the invention shown in FIG. 13, the front section of the product has at the waist region when viewed from the crotch region, concave recess 40 between the hip sections, here denoted by 41, 42. During product usage, the front section of the product, as can be seen from FIG. 13, hence acquires a low pants cut. Compared with a straight cut, this reduces the amount by which the upper limit edge 19 of the product in the middle region of the front section needs to be pulled down in order to bring the said limit edge into position below the penis of the user. The absorbent product can, for example, be provided at the said hip sections 41, 42 with friction members (not shown) in the form of friction glue or a foam material with high surface friction on the inner or outer side of the product. The friction members are intended to help the product remain wholly in place when the user pulls down the front section of the product in order to free his penis in connection with a toilet visit. In the embodiment according to FIG. 13, parts corresponding to similar components in other embodiments have been provided with the same reference notations. The upper limit edge 19 of the front section, after the latter has been pulled down, has been marked in FIG. 13 with a dash-dot line 19. The rear section 8 of the absorbent product is provided with waist elastic 20′.

The product according to an embodiment of the invention has a pants-like shape. The pants can be wholly or partially elastic with the same or different elastic properties in different regions. The elastic can be constituted by elastic non-woven material, elastic films or a combination of these materials. The elastic can also be produced by elastic threads or tape, which are fitted with the same or different pretensioning in different regions. The product expediently has a circumferential elastic waist region, which forms waist elastic for keeping the product in place on the user. The elastic member 20 on the front section of the product can constitute a section of the elastic waist region and can have the same or different elastic properties compared with the rest of the waist elastic. The essential point is that the elastic member allows the front section of the product to be pulled down without difficulty so that the upper limit edge of the product in the middle region of the front section is situated below the penis of the user.

Elastic film and elastic non-woven material can be configured such that it is elastic in more than one direction. Materials of this type can, of course, be used in the front section of the product over the whole or sections of this. Like elastic threads or elastic tape, films or non-wovens which are elastic in only one direction can also be used to form the elastic member.

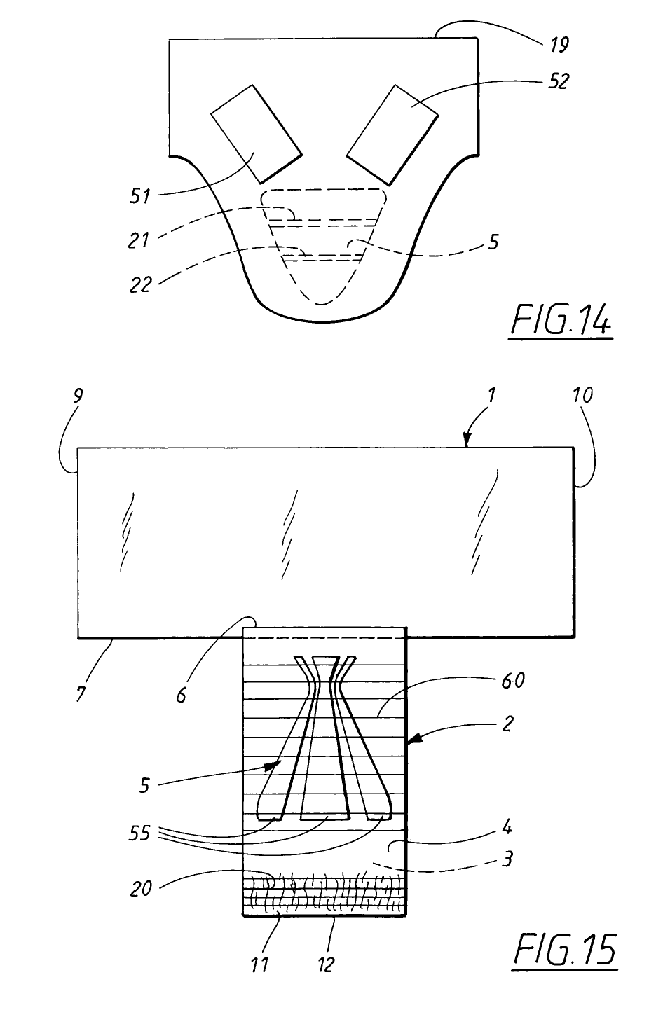

In FIG. 14, an illustrative embodiment of a product according to the invention is shown diagrammatically, having an elastic member formed by two zones 51, 52 of a film which is elastic in the one direction in the said zones. The direction of the elastic in the said zones is arranged such that this is effective in the longitudinal direction of the said zones. When the front section of the product is pulled down, the intended pulling down of the elastically stretchable zones is facilitated.

In the embodiment shown in FIG. 15, those parts corresponding to similar components in the embodiment according to FIG. 1 have been provided with the same reference numerals. The absorbent element 5 according to this embodiment is constituted by a number of separate absorption bodies 55 which are fitted adjacent to one another in the lateral direction and extend in the longitudinal direction of the product from the front section to the rear section and which are separable in the vertical direction and the lateral direction counter to the action of transverse elastic 60. The separate absorption bodies can hence be mutually displaced when the front section of the product is pulled down. The absorbent element is in this case configured correspondingly to the description given in WO 91/09580, to which allusion is made as a reference. The elastic member 20 has here been formed by a number of parallel elastic threads.

Although the invention has been described in connection with preferred embodiments thereof, it will be appreciated by those skilled in the art that additions, deletions, modifications, and substitutions not specifically described, are possible without departing from the spirit and scope of the invention as defined in the appended claims.

What is claimed is:

1. A pants-shaped absorbent product for males, comprising:
a front section;
a rear section;
a crotch section;
side sections to connect the front section and the rear section in the lateral direction, wherein the side sections comprise hip sections;
an elastic waist region;
an absorbent element, wherein the absorbent element at least covers the penis of the user; and
a liquid-tight outer layer to cover the absorbent element at least on the side thereof facing away from the user when the product is worn;
wherein the front section has at least one elastic member which enables the front section of the product to be pulled down during use, counter to the action of the elastic member, to a position in which the upper limit edge of the front section in the middle region of the front section is situated below the penis of the user, at the same time as at least the hip sections of the product are arranged to be held in place around the waist of the user;
wherein the absorbent element is configured with one or more deformation zones, which enable parts of the absorbent element which are situated above and over the penis of the user during product usage to be drawn down together with the rest of the front section when the front section of the product is pulled down; and
wherein the front section and the absorbent element are arranged to be returned by the elastic member to an original usage position.

2. The absorbent product of claim 1, wherein the elastic member, when stretched over the whole of a working range of the elastic member, has an essentially constant tensile stress when stretched in the same order of magnitude as the tensile stress in the elastic waist region.

3. The absorbent product of claim 1, wherein the elastic member comprises a section of the elastic waist region.

4. The absorbent product of claim 3, wherein the section of the elastic waist region extends over the front section between the hip sections.

5. The absorbent product of claim 4, wherein the elastic waist region extends over the whole of the front section, rear section, and side sections and wherein the section of the elastic waist region which serves as the elastic member has higher elastic stretchability than the rest of the elastic waist region.

6. The absorbent product of claim 1, wherein the absorbent element is of a thinner and more flexible configuration in the deformable zone compared with the rest of the absorbent element.

7. The absorbent product of claim 1, wherein the absorbent element comprises deformable zones in the form of fold notches for the absorbent element and wherein the absorbent product is bent along the fold notches when the front section is pulled down.

8. The absorbent product of claim 7, wherein the fold notches comprise recesses, through-slots, or compression lines in the absorbent element.

9. The absorbent product of claim 1, wherein the absorbent element comprises at least two absorption bodies of different extent, wherein only one of the absorption bodies extends into the deformation zone, and wherein the absorption body extending into the deformation zone has little or no inherent stiffness.

10. The absorbent product of claim 1, wherein the front section, at the waist region, when viewed from the crotch region, has a concave recess between the hip sections, whereby the front section of the product acquires a low pants cut during use, which reduces the amount by which the upper limit edge of the absorbent product in the middle region of the front section needs to be pulled down in order to bring the said limit edge into position below the penis of the user, compared with a straight cut.

11. The absorbent product of claim 1, wherein the hip sections comprise friction members arranged to help the absorbent product to be held in place on the user as the front section of the absorbent product is pulled down.

12. The absorbent product of claim 1, wherein the elastic waist region is formed at least in part from an elastic first piece, which is essentially rectangular in the extended state, to partially surround the trunk of the user and form the rear section and side sections comprising hip sections, wherein a second piece incorporated in the product is configured to form the front section and crotch section, wherein the second piece is elongated with two opposing end edges and two opposing longitudinal edges, wherein the width of the second piece, at least at the crotch section, is less than the length of the first piece, wherein the second piece is arranged in the longitudinal direction perpendicularly to the longitudinal direction of the first piece and is connected by a first end section centrally onto the one longitudinal edge section of the first piece, wherein the one end section of the first piece is connected to a first side edge section of the second piece, and wherein the second end section of the first piece is correspondingly connected to a second side edge section of the second piece and the absorbent element is fitted, in its entirety, on the second piece.

13. The absorbent product of claim 12, wherein the connected side edge sections and end sections of the said first and second pieces are fitted prior to connection in overlapping arrangement with the inner side of an overlapping section placed against the outer side of an overlapped section.

14. The absorbent product of claim 1, wherein the absorbent element extends in the longitudinal direction over the whole of the crotch section and slightly up over the front section in the direction of the upper side edge of the second piece during use.

15. The absorbent product of claim 1, wherein the elastic member comprises an elastic non-woven or an elastic film.

16. The absorbent product of claim 1, wherein the elastic member comprises elastic tape or threads.

17. The absorbent product of claim 1, wherein the absorbent product comprises a two-piece product having an absorbent element in the form of a detachable insert to be fitted inside pants incorporated in the product, wherein the insert has a rear liquid-tight layer provided with fastening members by means of which the insert is detachably anchored in the pants, and wherein the insert is arranged to be held in place relative to the pants by means of the fastening members when the front section of the product is pulled down.

18. The absorbent product of claim 1, wherein the absorbent element comprises a number of separate absorption bodies fitted adjacent to one another in the lateral direction and extending in the longitudinal direction of the product in the direction from the front section to the rear section and wherein the separate absorbent bodies are separable in the lateral direction and longitudinal direction counter to the action of transverse elastic, whereby the separate absorption bodies can be mutually displaced when the front section of the product is pulled down.
